(19) United States
(12) Patent Application Publication
(10) Pub. No.: US 2014/0121168 A1
(43) Pub. Date: May 1, 2014

(54) ANTAGONISTS OF TRPV1 RECEPTOR

(71) Applicants: Eric Schmidt, SALT LAKE CITY, UT (US); Alan R. Light, Salt Lake City, UT (US); Baldomero M. Olivera, Salt Lake City, UT (US); Christopher A. Reilly, Salt Lake City, UT (US); Zhejian Lin, Salt Lake City, UT (US); Gisela P. Concepcion, Quezon City (PH)

(72) Inventors: Eric Schmidt, SALT LAKE CITY, UT (US); Alan R. Light, Salt Lake City, UT (US); Baldomero M. Olivera, Salt Lake City, UT (US); Christopher A. Reilly, Salt Lake City, UT (US); Zhejian Lin, Salt Lake City, UT (US); Gisela P. Concepcion, Quezon City (PH)

(73) Assignee: ANTAGONISTS OF TRPV1 RECEPTOR, SALT LAKE CITY, UT (US)

(21) Appl. No.: 14/114,371

(22) PCT Filed: Apr. 26, 2012
(86) PCT No.: PCT/US12/35294

§ 371 Date: Jan. 8, 2014

Related U.S. Application Data
(60) Provisional application No. 61/517,802, filed on Apr. 26, 2011.

(51) Int. Cl. C07K 7/52; C07K 7/06

(52) U.S. Cl. CPC C07K 7/52; C07K 7/06
USPC 514/18.3; 530/317; 530/329

(57) ABSTRACT

TRPV1 antagonists and associated methods are provided. A TRPV1 channel antagonist can have the structure: Formula (I) wherein R₁ can be —CH₃, —(CH₂)_{X}(CH)_{Y}CH₃ where x+y=1-20, an aromatic, a (CH₂)ₙ aromatic where n can be less than or equal to 6, a lipid, or a linker, and wherein R₂ can be either Formula (II) or Formula (III) Additionally, R₃ can be —O—R₄ or —NH—R₄ and R₄ can be —H, —CH₃, an ester, a cyclic ester, or an amide.

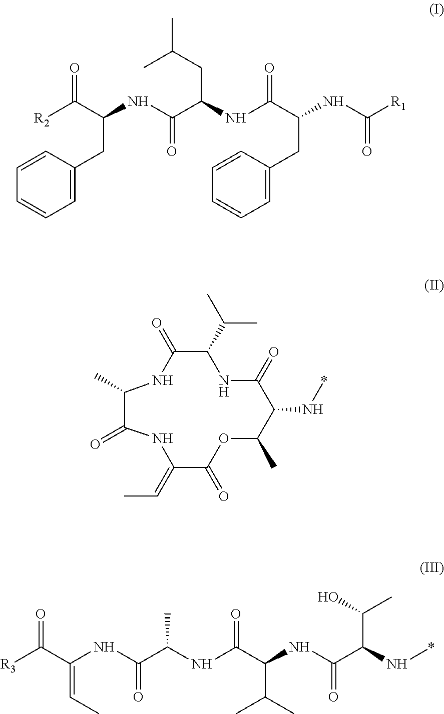

### RELATED APPLICATION

This application claims the benefit of U.S. Provisional Application Ser. No. 61/517,802, filed on Apr. 26, 2011, which is incorporated herein by reference.

### GOVERNMENT INTEREST

This invention was made with government support under ICBG Grant No. U01TW008163 from Fogarty (NIH), NIH Grant ES01734. The United States government has certain rights to this invention.

### BACKGROUND OF THE INVENTION

More than thirty Transient Receptor Potential (TRP) channels are known, including many that are important in sensing stimuli such as cold, heat, and pain. Among these, TRP Vanilloid-1 (TRPV1; VR1) is a nonselective cation channel that is a major mediator of pain and inflammation. Stimuli such as heat, protons, and chemical ligands provoke action potentials, leading to the release of neurotransmitters and neuroactive peptides (e.g., substance P, neurokinin A, and CGRP) from peripheral and central nerve terminals. Many lines of experimental evidence indicate that selective TRPV1 antagonism could play a useful role in the treatment of chronic pain and inflammatory hyperalgesia, and a variety of ligands have been reported. Endovanilloids and the endogenously supplied ligand, capsaicin, are potent TRPV1 activators that cause sensations of heat and pain in the short term, but lead to pain desensitization in the longer term. Vanilloids such as capsaicin bind at an intramembrane and intracellular site located between TM segments 3 and 4, involving Y511, S512, W549, and other residues. This site is distinct from the channel pore-loop segment, but binding of ligands to this site is presumed to induce structural changes in the pore loop region such that ions can pass through the tetrameric receptor pore.

### SUMMARY OF THE INVENTION

TRPV1 antagonists and associated methods are provided. In one aspect, for example, a TRPV1 channel antagonist can have the structure:

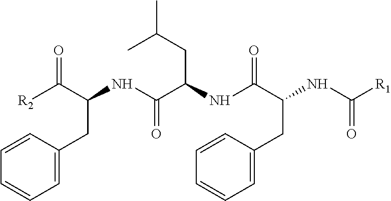

wherein R₁ can be —CH₃, —(CH₂)ₓ(CH)_{y}CH₃ where x+y=1-20, an aromatic, a (CH₂)ₙ aromatic where n can be less than or equal to 6, a lipid, or a linker, and wherein R₂ can be either

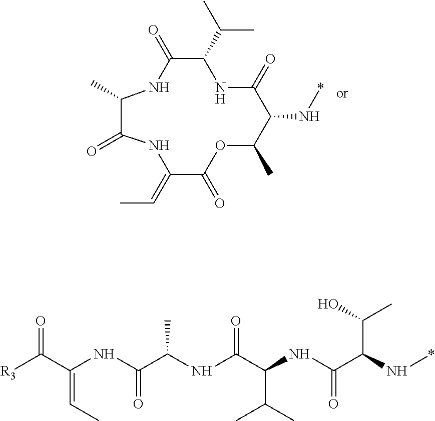

Additionally, R₃ can be —O—R₄ or —NH—R₄ and R₄ can be —H, —CH₃, an ester, a cyclic ester, or an amide.

In another aspect, a method of antagonistically blocking a TRPV1 channel is provided. Such a method can include delivering a TRPV1 channel antagonist to the TRPV1 channel, wherein the TRPV1 channel antagonist has the structure:

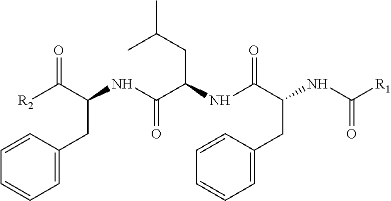

where R₁ can be —CH₃, —CH₂CH₃, —CH₂CH₂CH₃, —CH₂CH₂CH₂CH₃, —CH₂CH₂CH₂CH₂CH₃, —CH₂CH₂CH₂CH₂CH₂CH₃, a lipid, or a linker, and where R₂ can be either

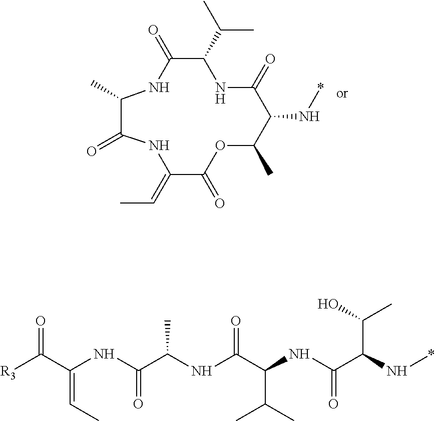

Additionally, R₃ can be —O—R₄ or —NH—R₄ and R₄ can be —H, —CH₃, an ester, a cyclic ester, or an amide.

In yet another aspect, a method of protecting sensitive areas of a human from effects associated with a TRPV1 channel agonist exposure is provided. Such a method can include applying to the sensitive areas a composition comprising the TRPV1 channel antagonist according to the present aspects in a physiologically acceptable carrier. In one specific aspect, the TRPV1 channel agonist is capsaicin.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

Additional features and advantages of the invention will be apparent from the detailed description which follows, taken in conjunction with the accompanying drawings, which together illustrate, by way of example, features of the disclosure.

FIG. 1 shows chemical structures of compounds 1, 1a, and 2-11 according to one aspect of the present disclosure;

FIG. 2 is graphical representation of data according to another aspect of the present disclosure;

FIG. 3 is graphical representation of data according to another aspect of the present disclosure; and

FIG. 4 is graphical representation of data according to another aspect of the present disclosure.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and “the” include plural referents unless the context clearly dictates otherwise. Thus, for example, reference to “a receptor” includes one or more of such receptors, and reference to “the channel” includes reference to one or more of such channels.

### DEFINITIONS

In describing and claiming the present invention, the following terminology will be used in accordance with the definitions set forth below.

As used herein, “subject” refers to humans, and can also include other animals such as horses, pigs, cattle, dogs, cats, rabbits, and aquatic mammals.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

TRPV1 Antagonists

As has been described, Transient Receptor Potential Vanilloid-1 (TRPV1) is a nonselective cation channel that can be activated by a wide variety of exogenous and endogenous physical and chemical stimuli. For example, exogenous stimuli that can activate TRPV1 can include heat greater than about 43° C., capsaicin, the pungent compound in hot chili peppers, allyl isothiocyanate, the pungent compound in mustard and wasabi, and the like. The activation of TRPV1 leads to a painful, burning sensation. Endogenous activators of TRPV1 can include low pH, the endocannabinoid anandamide, N-arachidonoyl-dopamine, and the like. TRPV1 receptors are found in the nociceptive neurons of the peripheral nervous system, but have also been described in various other tissues, including the central nervous system. Thus, TRPV1 is involved in the transmission and modulation of pain as well as the integration of diverse painful stimuli.

TRPV1 receptors are found in dorsal root ganglion (DRG) neurons, which transfer afferent signals such as pain, heat, and touch. These neurons also contain various other types of channels and receptors, making them useful models for broad-net drug discovery assays. Such an assay has now been employed using a primary culture of mouse DRG neurons containing many of the different cell types normally present in the DRG of live mice. These cells are exposed to a series of treatments including KCl and chemical extracts or pure compounds. By observing differences in the resulting Ca²⁺ flux, compounds have been discovered that act directly on channels and receptors important in transferring information about pain, heat, touch, and other properties.

Various compounds have been discovered that function as antagonists to TRPV1 channels. In one aspect, for example, a TRPV1 channel antagonist is provided as shown in Formula I:

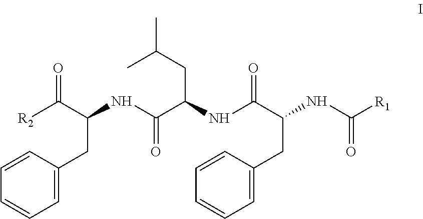

where R₁ can be —CH₃, —(CH₂)ₓ(CH)_{y}CH₃ where x+y=1-20, an aromatic, a (CH₂)ₙ aromatic where n is less than or equal to 6, a lipid, or a linker. Thus, the carbon groups of R₁ can be linear or branched.

Non-limiting examples of hydrocarbon groups can include methyl, ethyl, propyl, isopropyl, butyl, isobutyl, tert-butyl, pentanoyl, hexanoyl, heptanoyl, octanoyl, nonanoyl, decanoyl, lauryl, myristyl, palmitoyl, stearoyl, palmitoleoyl, stearoyl, arachidonoyl, isoprenyl, farnesyl, geranyl, angelyl, aminomethyl, hydroxymethyl, thiomethyl, aminoethyl, hydroxyethyl, thioethyl, aminobutyl, hydroxybutyl, thiobutyl. Non-limiting examples of alkyl aromatic groups can include benzyl, phenyl, biphenyl, triphenyl, indolyl, furanyl, thiophenyl, pyridinyl, pyranyl, bypyridyl, imidazolyl, triazolyl, napthylenyl. Suitable groups and derivatives can be selected based on binding kinetics to TRPV1 such that the antagonist binds with the receptor over a long period of time (e.g. over one to two hours). These groups are also chosen for compatibility with the application area. For example, derivatives can be selected that have lower binding constants to TRPV1 than the parent compounds, or those that effect whether or not TRPV1 is internalized in cells. Other desired properties of such derivatives include improved solubility and decreased toxicity in comparison to the parent compounds, or targeting the molecules to specific tissues or cell types.

Lipids can include a variety of chemical compounds, and are well known in the art. Non-limiting examples of such lipids can include methyl, ethyl, propyl, benzyl, cypionyl, phenyl, aromatic, hydroxyethyl, and combinations thereof. Lipid groups can be formed and used in a variety of ways. For example, acyl groups having various chain length, branching, unsaturation, and substitution can be used. R1 is attached to the acyl group that would be part of a fatty acid, so that R1=methyl derives from acetate, etc. However, the core pharmacophore is within the underlying peptide structure, such that these acyl groups are substantially less influential on function of the molecule than other groups. Diverse functional groups can also be added to this position, including various lengths of acyl chains, aromatic derivatives, fluorescent derivatives, PEG-based linkers, and the like.

Linkers can include a variety of chemical compounds that are commonly used in the art. Non-limiting examples include polyethylene glycol, polyamines, ethanolamines, alkyl chains, spermidine, and combinations thereof. Linkers include bridging substituents that link the active portion of the molecule with carriers, solubility enhancers, targeting moieties, fluorescent probes, antibodies, proteins, solid resins, nanoparticles, inorganic carriers, and similar substituents. Linkers can also increase the multiplicity of the drug by covalently linking two or more of the active substituents together.

The R₂ group can include a variety of moieties. In one aspect, for example, R₂ can include the group shown in Formula II such that the TRPV1 channel antagonist can have the structure shown in Formula III:

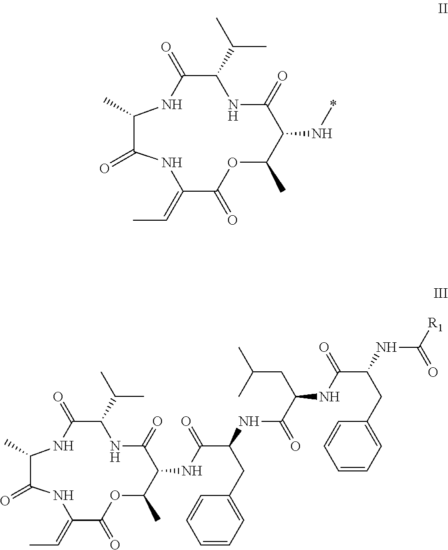

In another aspect, R₂ can include the group shown in Formula IV such that the TRPV1 channel antagonist has the structure shown in Formula V:

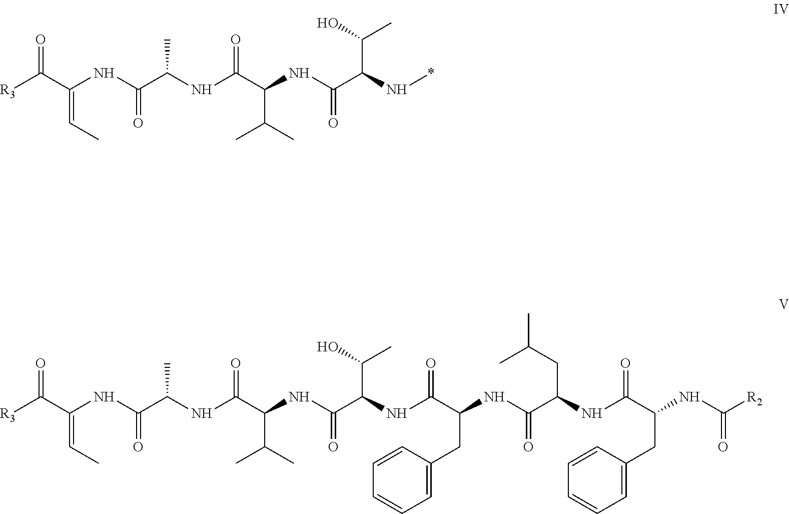

In the case of Formulas IV and V, R₃ can be —O—R₄ or —NH—R₄, and R₄ can be —H, —CH₃, an ester, a cyclic ester, an amide, or the like. Any amide can be utilized, however in one aspect the amide can have from 0 to 7 carbons. Amides can also avoid hindrance to reaching the active site, can be activated in vivo to reach the active site.

In another aspect, a method of antagonistically blocking a TRPV1 channel is provided. Such a method can include delivering a TRPV1 channel antagonist to the TRPV1 channel, where the TRPV1 channel antagonist has a structure according to aspects of the present disclosure. Blocking TRPV1 channels can have a variety of benefits to a subject, and it is understood that any treatment use derived therefrom is considered to be within the present scope. For example, in one aspect blocking TRPV1 activity can be used to reduce pain. Various forms of and neuropathic pain can be treated, including without limitation, pain associated with multiple sclerosis, chemotherapy, amputation, and the like, as well as pain associated with the inflammatory response of damaged tissue. One example of such an inflammatory condition can include osteoarthritis. TRPV1 antagonists can also be utilized to treat a subject that has been exposed to a TRPV1 agonist such as capsaicin, allyl isothiocyanate, or the like. TRPV1 is also expressed in the central nervous system (CNS), and thus TRPV1 antagonists can be utilized to treat various CNS conditions, such as, for example, anxiety, memory conditions, epilepsy, and the like. In some cases, TRPV1 antagonists may be utilized to regulate temperature in certain subjects.

In another aspect, a method of protecting sensitive areas of a human from the effects associated with a TRPV1 channel agonist exposure is provided. Such a method can include applying at least to the sensitive areas a composition comprising the TRPV1 channel antagonist according to aspects of the present disclosure in a physiologically acceptable carrier. In such cases, the antagonist can bind to the TRPV1 channel, thus providing protection to the human against a subsequent agonist exposure from, for example, capsaicin.

For the purposes of delivery of a TRPV1 antagonist to a subject, the antagonist can be dispersed in a physiologically acceptable carrier. Such carriers are well known in the art, and any carrier capable of containing and delivering the antagonist to the subject is considered to be within the present scope. Furthermore, the carrier can vary depending on the delivery mode of the composition, the area to which the composition is to be delivered, and the condition being treated. Non-limiting examples of such carriers can include liposomes, proteins, synthetic polymers, and nanoparticles.

Additionally, the antagonist can be present in the carrier in a therapeutically effective amount or in an amount sufficient to provide protection. As such a broad range of concentrations of TRPV1 antagonists in a carrier is contemplated. In one aspect, for example, the TRPV1 channel antagonist is present in the physiologically acceptable carrier at a concentration of from about 10 to about 1000 micromolar.

The TRPV1 antagonist composition can be delivered to a subject via a variety of mechanisms. Any delivery mechanism capable of applying the antagonist to TRPV1 receptors is considered to be within the present scope. In one aspect, for example, the composition can be topically delivered to act at or near the surface of the area being treated. Such a topical delivery composition can include, without limitation, sprays, lotions, gels, ointments, patches, buccal applicators, ocular applicators, nasal applicators, and the like, including combinations thereof. In another aspect, the composition can be invasively delivered for localized or systemic treatment. Non-limiting examples of such invasive delivery can include intravenous, intramuscular, subcutaneous, and the like, including combinations thereof. Furthermore, the composition can be orally delivered in the form of a tablet, pill, gelcap, liquid, gum, chew, sucker, lozenge, or any other oral delivery mechanism. It is also contemplated that the composition can be delivered by iontophoretic transdermal delivery.

TRPV1 antagonists can be particularly suited to treating pain. Additional indications can include fatigue, low core temperature, and increasing exercise performance. These compounds can also be used to decrease complications caused by sepsis, and complications in inflammatory lung diseases. As mentioned above, the TRPV1 antagonist compositions can be used to provide topical relief to burns or abrasions, for example.

The following is a description of the isolation of various TRPV1 antagonists and subsequent testing using various assays. It is noted that this is exemplary, and that the present scope should not be limited to the specific compounds disclosed or the assays utilized.

A Dorsal Root Ganglion (DRG) assay can be beneficial for studies of channel pharmacology. One of the advantages of a DRG assay is that cell types containing distinct receptor and channel populations can be pharmacologically distinguished. For instance, application of capsaicin differentiates nociceptors from other cell types. As one example, in the course of screening bacterial extracts for DRG activity, the organic extract of strain Streptomyces sp. CN48 produced a novel effect; in addition to strongly increasing Ca²⁺ in DRG neurons in response to KCl addition, the extract inactivated all response to capsaicin, even 2 min after removal of the extract. It is thus likely that this extract may inactivate TRPV1 through a novel mechanism.

A variety of peptides have thus been discovered that produce that produce long-term (>1 h) inhibition of TRPV1. Their effects on endogenously expressed and recombinant wild-type and mutant human TRPV1 channels are assessed, showing that the compounds function by a mechanism that appears to involve covalent modification of TRPV1 through residues that constitute an intracellular helix spanning TM4 and 5 and the pore-loop segment.

Strains CN48 and CT3a are cultivated from dissected tissues of the mollusks Chicoreus nobilis and Conus tribblei, respectively, from Cebu, Philippines. 16S gene sequence analyses shows that both strains belong to the genus Streptomyces. Sequences are deposited in GenBank, accession numbers, HQ696493 (CN48), HQ696492 (CT3a).

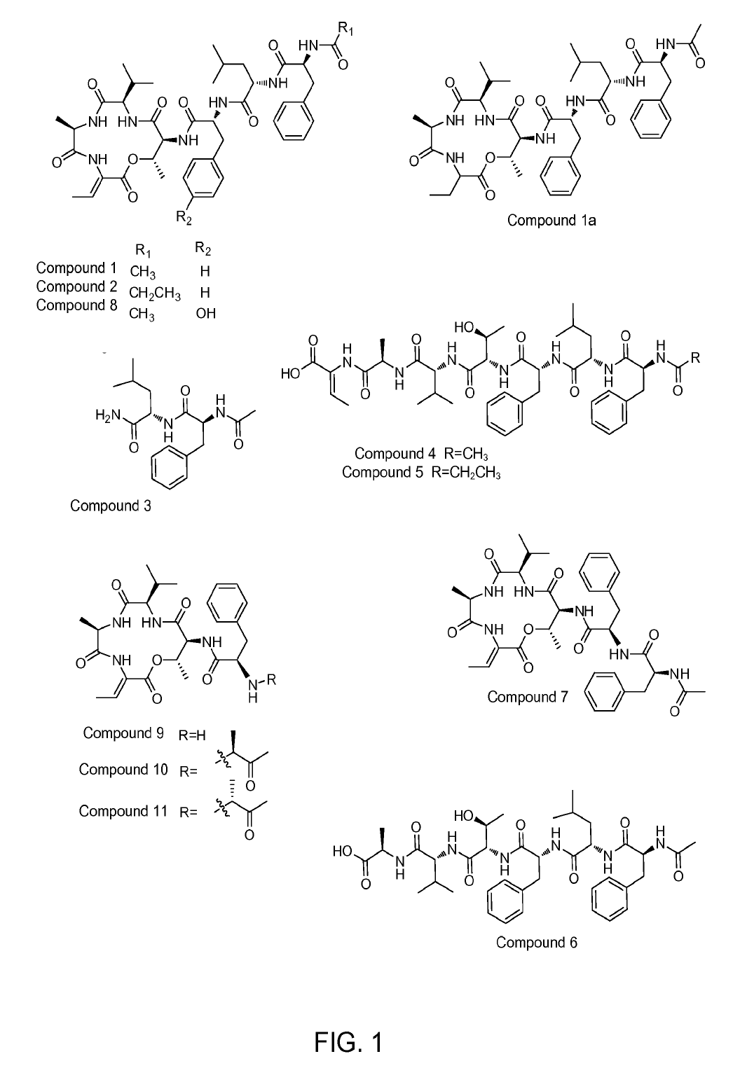

Crude extracts of CN48 and CT3a are strongly active in the DRG assay. The CN48 extract strongly activated DRG cells upon addition of KCl, while that from CT3a is strongly deactivating. Moreover, the CN48 extract blocks activation by capsaicin. Despite these differences, HPLC analysis shows that the two strains contain closely related families of metabolites, which are further purified by bioassay- and chemistry-guided fractionation. Following fermentation, cells are pelleted by centrifugation, and the resulting broths are subjected to HP20 resin adsorption chromatography. The moderately polar fractions are further purified by C₁₈ flash chromatography followed by C₁₈ HPLC to yield compounds 1, 1a, and 2-11 (see FIG. 1).

By comparison of spectroscopic data and physicochemical properties with literature reports, compounds 1 and 2 are identified as the previously reported metabolites, A-3302-B (also known as TL-119) and A-3302-A. Isolated compound 3, N-acetyl-L-phenylalanyl-L-leucinamide, has been synthesized but is not previously known as a natural product; in this case compound 3 is instead composed of D-amino acids.

The molecular formula of compound 4 is determined as C₄₂H₅₉N₇O₁₀ by high-resolution electrospray ionization mass spectrometry (HRESIMS) and ¹H and ¹³C NMR data (See Tables 1 and 2), indicating that it is larger than compound 1 by H₂O. In addition, the NMR data of compound 4 are similar to those of compound 1, with the main difference being the threonine β-methine ¹H and ¹³C resonances of compound 4 are shifted 0.8 ppm and 5 ppm upfield in comparison to those of compound 1. This difference suggests that compound 4 is a linear peptide lacking the lactone linkage found in compound 1. To confirm this hypothesis, a complete analysis of 1D and 2D NMR data was performed, showing that compound 4 contains the same amino acid residues in the same order as compound 1: α,β-dehydrobutyrine (Dhb), alanine (Ala), valine (Val), threonine (Thr), leucine (Leu), and two phenylalanines (Phe). Additionally, like compound 1, compound 4 is acetylated at its N-terminus.

Compound 5 was assigned the molecular formula C₄₃H₆₁N₇O₁₀ on the basis of HRESIMS analysis and NMR experiments (Tables 1 and 2), making it larger than compound 2 by H₂O and larger than compound 4 by CH₂. Detailed analysis of ¹H-¹H COSY and HMBC NMR experiments indicates that compound 5 differs from compound 4 only by the absence of an acetyl group; instead, a propionyl group (δ_{H} 1.98, 2H; 0.82, 3H) is assigned to compound 5. Analysis of 2D NMR data of compound 5 confirmed the presence of propionyl group and defined the amino acid sequence. Compound 5 is similar to compound 2, except that it is linear instead of a cyclic depsipeptide. Although compound 4 and compound 5 are derivatives of compounds 1 and 2, careful HPLC analysis indicates that they are indeed present in the fermentation media during the normal course of bacterial growth in the same ratios found after chemical isolation. Thus, they are naturally produced by these strains under these experimental conditions and are not extraction artifacts.

Compound 6 was found to possess the molecular formula C₃₈H₅₄N₆O₉ by analysis of HRESIMS and NMR data (Tables 1 and 2). The difference in the molecular formula of C₄H₅NO from compound 4 is attributed to the absence of the Dhb residue. The ¹H NMR of compound 6 lacks the olefinic proton signal at about 6.5 ppm found in compound 4 and compound 5. Analysis of NMR data confirmed the absence of the Dhb residue and allowed assignment of the amino acid sequence for compound 6.

Compound 7 was assigned the molecular formula C₃₆H₄₆N₆O₈ on the basis of HRESIMS analysis and NMR experiments (Tables 1 and 2). The NMR data of compound 7 are related to those of compound 1. The chemical shifts of the Thr β-methine group are observed at δ_{H} 4.72 and δ_{C} 73.9, which suggests that compound 7 is a cyclic peptide with a lactone linkage between the C-terminal amino acid and the hydroxyl group of the Thr residue. Further analysis of the NMR data of compound 7 shows that, in comparison to compound 1, Leu is absent. A NOESY correlation between protons at 4.48 ppm and 8.42 ppm establishes the connection between the two Phe residues in place of Leu.

The molecular formula of compound 8 was determined as C₄₂H₅₇N₇O₁₀ by high resolution ESIMS coupled with ¹H and ¹³C NMR data (Tables 1 and 2). The difference in the molecular formula of an oxygen atom from compound 1 is attributed to the presence of a tyrosine (Tyr) residue in place of Phe. The ¹H NMR spectrum of compound 8 shows a pair of aromatic doublets δ_{H} 7.00, 6.62, which are assigned to Tyr. The ¹³C NMR spectrum (Table 2) of compound 8 also indicates an oxygenated phenyl carbon at δ_{C} 156.5. HMBC and NOESY NMR data are consistent with the proposed structure of compound 8.

Compound 9 was assigned the molecular formula C₂₅H₃₅N₅O₆ on the basis of HRESIMS analysis and NMR experiments (Tables 1 and 2). The difference in the molecular weight in comparison to compound 1 is attributed to the absence of two amino acid residues (Leu and Phe) and an acetyl group from the N-terminus of the peptide. Compatibly, the NMR data (Tables 1 and 2) of compound 9 shows the absence of those signals for the two amino acid residues and acetyl group. Analysis of HMBC and NOESY NMR data confirmed the amino acid sequence of compound 9.

Compounds 10 and 11 are isolated with very similar HPLC retention times. The molecular formula for both was determined as C₂₉H₄₁N₅O₇ by high resolution ESIMS coupled with NMR data (Tables 1 and 2). The ¹H and ¹³C NMR data of compound 10 and compound 11 show similar NMR signals (Tables 1 and 2), suggesting they might be isomers of each other. In comparison the ¹H and ¹³C NMR data (Tables 1 and 2) to that of compound 9, two more methyl groups (δ_{H} 2.14, H-3, δ_{C} 27.8, C-3 and δ_{H} 1.17, H-4, δ_{C} 19.3, C-4) and a methine group (δ_{H} 3.42, H-1, δ_{C} 64.7, C-1) are present. The distinctive methyl singlet at δ_{H} 2.14 corresponds to an α-keto methyl group, but no ketone carbon is observed in the ¹³C NMR spectrum (Table 2) of compound 10. In the HMBC spectrum of compound 10, two strong correlations are observed from both H-3 and H-4 to a ketone carbon at 215.3 ppm, indicating a —CH(CH₃)COCH₃ fragment. Further HMBC correlations from H-1 to the α-C (δ_{C} 64.9) of Phe, and from the α-H (δ_{H} 3.30) of Phe to C-1 indicates the connection of these two partial structures. The same features are found in the NMR data of compound 11. Therefore, compound 11 has the same planar structure as compound 10.

Configurations are assigned using Marfey's method. Compounds 4-11 are hydrolyzed, and the resulting amino acids are converted to Nα-(2,4-dinitro-5-fluorophenyl)-L-alaninamide derivatives, which are characterized by HPLC in comparison with authentic standards. Compounds 4-6 contain L-Ala, L-Val, D-allo-Thr, D-Leu, and L- and D-Phe. In comparison to 4-6; compound 7 is similar but lacks D-Leu; compound 8 contains L-Tyr in place of L-Phe; and compounds 9-11 lack D-Phe and D-Leu. Compound 3 consists of D-Leu-D-Phe. When both L- and D-Phe are present, the order can not be ascertained from these experiments. However, compound 1 has been found in both the cultures of CT3a and CN48. It is thus proposed that the configurations of compounds 1-11 are similar. This hypothesis is further supported by comparison with the configurations of compounds 7-11, which are completely defined experimentally and in which only one Phe residue is present.

Compounds 1-11 are related to each other, belonging to a family that was previously only known from Bacillus subtilis. There are a few noteworthy modifications. Compound 6 is related to compound 4 by the loss of Abu; this may be due to enzymatic hydrolysis post-synthesis or to imperfect product synthesis by biosynthetic enzymes. In comparison to compound 1, compound 7 is missing a Leu that is within the peptide sequence itself. If the compounds are indeed produced nonribosomally, this may be due to module skipping. Compounds 10 and 11 are related to the rest by loss of the two N-terminal amino acids, which are instead replaced by an ketone derivative.

A fluorometric calcium flux DRG assay allows for the simultaneous study of 100-150 neurons with single application of compounds. Multiple neuronal cell types are present in each well, including a substantial fraction of nociceptors (˜30-50%); each cell type has different combinations of receptors and channels and exhibits a different pharmacological profile. In the standard discovery assay, responses elicited by chemicals are normalized by pulsing with 25 mM KCl, which leads to depolarization, followed by washout, bringing cells back to baseline. Subsequently, samples of extracts or pure compounds are added to observe any direct depolarizing effects, and then samples are added in tandem with 25 mM KCl to observe any increase or decrease of depolarization. After a washout period, capsaicin (a TRPV1 agonist) is added to differentiate nociceptors from other cell types. Finally, a pulse of 100 mM KCl is added to determine whether cells are still responding normally and to obtain a value for maximum depolarization. By following changes in intracellular Ca²⁺ over these steps, fine information about the activity of extracts is revealed, enabling discovery of new agents.

Using the above procedure, application of a major fraction of Streptomyces sp. CN48 extract led to a complete loss of response to capsaicin, but subsequent addition of 100 mM KCl still strongly depolarized all neurons in assay wells, indicating that they otherwise were alive and functioning normally. In addition, the extract was mildly stimulating to cells when co-applied with 25 mM KCl. In a pure compound test, 5 min after application of purified compound 4 at a final concentration of 125 μM, the DRG cells were depolarized by 25 mM KCl, and after that a complete loss of response to capsaicin was observed. Based upon these results, it is proposed that the nobilamides in the CN48 extract are responsible both for increased depolarization of DRG cells and for inhibition of response to capsaicin.

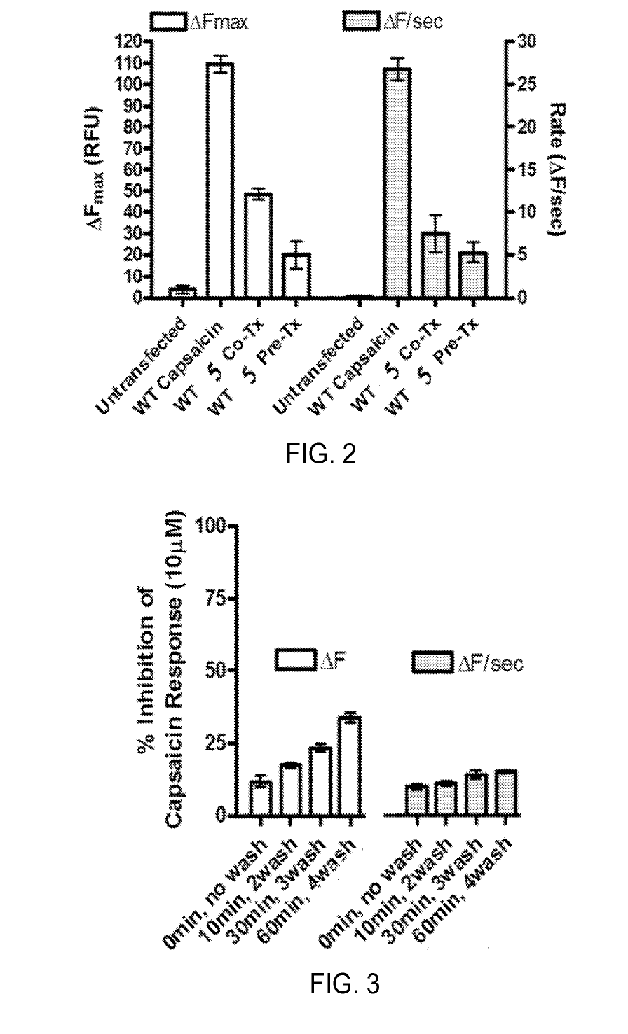

Two likely mechanisms can explain the DRG results. In the first, nobilamides (compounds 1-11) could impact regulatory proteins that reduce capsaicin receptor activity. In the second, due to the long delay prior to addition of capsaicin, it is possible that nobilimides irreversibly inactivate capsaicin receptors. Individual compounds were thus tested in assays using human bronchial epithelial BEAS-2B cells, which stably overexpress human TRPV1, primarily intracellularly. Direct competition experiments indicate that compounds 2 and 5 antagonize the action of capsaicin on TRPV1, with an apparent preference for cell-surface localized channels. Onset of action was slow, and pre-incubation led to substantially greater activity versus co-application. However, with this cell line it is difficult to determine whether this delayed effect might be due to a slow on-rate (such as found in some irreversible inhibitors) or whether cell penetration was a limiting factor for these large peptides. Therefore, HEK-293 cells were transfected with human TRPV1. This cell line does not normally express capsaicin-sensitive channels, and upon transfection TRPV1 is expressed largely on the cell surface, making it possible to measure inhibition in the absence of potential confounding effects. In this cell line, the potency of compound 5 was greatly increased with pre-incubation of the compounds prior to capsaicin application, in comparison to co-application with capsaicin (See FIG. 2). FIG. 2 shows time-dependent inhibition of TRPV1 by compound 5. HEK-293 cells are transfected with human wild type TRPV1 and pre-treated with compound 5 (375 μM), followed by application of capsaicin (25 μM). For comparison, cells are co-treated with both agents. The change in fluorescent response to Ca²⁺ (ΔF) is measured as a function of maximum fluorescence or of fluorescence rate (n=3).

This observation is consistent with an irreversible binding model. In a series of further experiments, the inhibitory activity of compound 5 was found to be stable through four washes taking place over a 60-min period (see FIG. 3). FIG. 3. shows the stability of TRPV1 inhibition. Compound 2 (375 μM) is incubated with HEK-293 cells transfected with wild-type human TRPV1. Response to capsaicin is measured over a 1 h time course with washes at various intervals (x-axis). The change in fluorescent response to Ca²⁺ (ΔF) is measured as a function of maximum fluorescence or of fluorescence rate. However, a slight rebound of activity was noticed after one hour in these human cells, indicating either a slow reversibility or replacement of inhibited TRPV1 by newly synthesized protein.

To further examine this effect, the long-term antagonism of mouse DRG neurons was investigated in an assay adapted to allow lengthy survival and monitoring of capsaicin responses in individual neurons (100-150 per well). In the first experiment, mouse DRG neurons are incubated with compound 4, then allowed to recover over a three-hour period. Although the capsaicin response is initially abolished, recovery is observed after three hours. To determine whether recovery is due to new protein synthesis, TRPV1 recycling, or slow reversibility of binding, cells are also treated with actinomycin D, cycloheximide, and brefeldin A under several different conditions to inhibit transcription, translation, and trafficking of TRPV1 to the cell surface and subjected to the same 3-hour recovery experiment. Individual neurons are followed for the entire duration of the experiment so that recovery of single cells can be observed. Response to capsaicin is restored after about three hours, indicating that channel synthesis or turnover is likely not involved in recovery, and that instead tightly or covalently bound compound 4 is slowly released over this time course. Thus, it can be concluded that nobilamides are long-acting antagonists of TRPV1, probably through a covalent modification of the receptor. Because agonists that act for >15 min on TRPV1 have previously been termed “essentially irreversible”, these nobilamides represent a class of essentially irreversible antagonists.

Because the inhibitory nobilamides contain the modest electrophile dehydrobutyrine, it appears that the compounds covalently modify nucleophiles in TRPV1, leading to inhibition. Indeed, the dehydrobutyrine residue of compound 1 is reduced with H₂, and the resulting compound 1a is completely inactive. Candidate nucleophiles include cysteine (Cys) residues in the pore loop region, which were previously shown to be important redox-sensitive residues. TRPV1 is very active in reducing conditions, and its activity greatly decreases through Cys disulfide formation in the channel under more oxidizing conditions. In addition, other TRP channels have been found that are agonized or antagonized by relatively non-specific electrophiles.

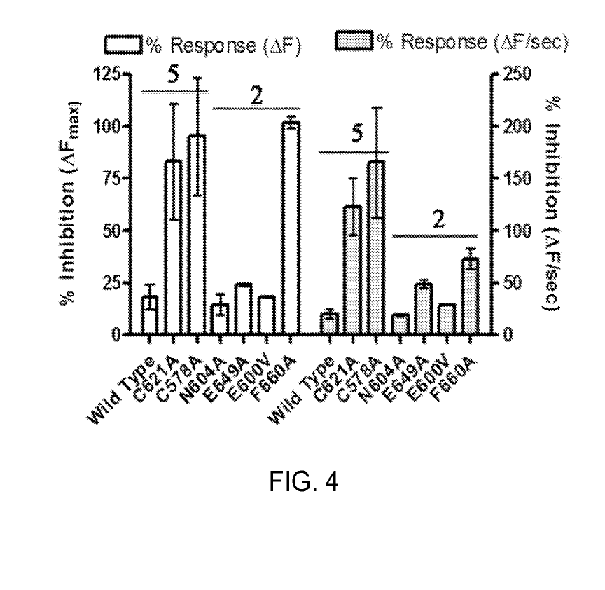

To test the site of binding and the possibility of covalent modification, HEK-239 cells overexpressing mutant human TRPV1 variants are treated with compounds 2 and 5 (see FIG. 4). Six mutants were used. Of these, three are inhibited by compounds 2 and 5 in a manner similar to wild type, while three exhibited significantly reduced inhibition by compounds 2 or 5. Two Cys mutants (C578A and C621A) and the F660A mutant, adjacent to the ion conducting pore of TRPV1, led to a complete loss of TRPV1 antagonism. These results suggest that compounds 2 and 5 bind directly to the pore-loop segment of the channel, where they act as channel blockers by either modifying local protein dynamics required for activation and/or ion flux or as a “molecular blockade” via the formation of covalent adducts that possibly bridge TRPV1 subunits to prevent ion flux from occurring. It is curious that removing either Cys residue abolishes the antagonist response, which defies simple models of single alkylation. Moreover, incubation of nobilamides with glutathione did not impact activity, indicating that the dehydrobutyrine residue is at most a modestly active electrophile that requires appropriate steric directing by adjacent residues such as F660 on TRPV1. It is thus proposed that nobilamides covalently inactivate TRPV1 based upon: 1) the lack of activity when the electrophile Dhb was reduced or absent; 2) the requirement for Cys residues in the binding site; 3) the slow onset time for activity; and 4) the long duration of binding.

FIG. 4 shows mutational analysis of human TRPV1 inhibition. FIG. 4A shows compounds 2 (375 μM) and 5 (375 μM) pre-incubated with HEK-293 cells transfected with wild-type and mutant TRPV1 channels, and inhibition of the capsaicin response is compared. The change in fluorescent response to Ca²⁺ (ΔF) is measured as a function of maximum fluorescence or of fluorescence rate.

Covalent Cys modification can be important for rodent TRPV1 agonists such as allicin and nitric oxide, which act primarily on C157 of the rat proteins. These activating effects are relatively short lived and readily reversible in comparison to the inactivating effect of nobilamides, which are long lasting and act in a wholly different region of the protein. By further contrast, a pore-loop Cys residue (C621) impacting nobilamide activity in human TRPV1 potentiates the response of the rat channel to heat. F660 is required for acid sensitivity, and mutations of this residue abolishes this response while maintaining capsaicin sensitivity. Recently, data were obtained indicating that F660 is probably primarily involved in gating the response to protons. It is interesting that nobilamides apparently directly act on or are affected by amino acids that potentiate the response to important additional TRPV1 activators. Based upon these results, the nobilamides appear to block the pore such that they antagonize activation by many different activators including capsaicin, heat, and protons. Direct channel blocking antagonists, such as tetrabutylammonium, have been previously described, but these in general exhibit lower potency than nobilamides, lack selectivity, and are readily reversible.

Persistent human and mouse TRPV1 antagonism was observed at relatively high nobilamide concentrations (above about 200 μM, in comparison to an activating capsaicin concentration of 2-25 μM in these cell lines). However, this activity is highly selective, and slight differences in structure can lead to complete abrogation of activity. Of these compounds, 1, 2, 4, and 5 exhibit substantial inhibitory activity. In fact, slight modifications lead to large activity differences. Compounds 1 and 4 exhibit relatively low activity, with IC₅₀ values of 1320 and 1665 μM, respectively. However, compounds 2 and 5 are much more potent, with IC₅₀ values of 227 and 275 μM, respectively. The main difference between compounds 2/5 and 1/4 is that the former are longer by a single methylene group. By contrast, the existence of a constraining ester does not appear to be highly important in conferring activity. Neither compound 6 nor compound 1a exhibited detectable activity, indicating the importance of the intact dehydrobutyrine residue. Additionally, compounds 7-11, which differ primarily by length or residue in the side chain, are totally inactive. Strikingly, the main difference between active compound 1 and inactive compound 8 was the presence of OH (Tyr) in place of H (Phe) in the side chain. Compound 7 is lacking a single Leu residue in the side chain. These results reinforce the selective nature of inactivation and indicate that inhibition is not due to a simple and nonspecific covalent modification.

As such, these results demonstrate that nobilamides antagonize TRPV1 and highlight the potent analogs that block the TRPV1 channel in this novel manner. Very slight differences in amino acids or in the N-terminus of the peptides exert large effects on activity. Moreover, within the natural derivatives the amino acid sequence is relatively fixed; a wide variety of modifications can be made by making substitutions. The presence of D-amino acids in key positions can also desirable for potent analogs, since D-peptides are typically more stable than their L-counterparts in human use. Finally, irreversibility itself may prove to be an advantage.

TABLE 1
¹H (500 MHz) NMR Data for Nobilamides A-H (4-11) in DMSO-d6.

| | | 4 | 5 | 6 | 7 | 8 | 9 | | 10^{a} | 11^{a} |
|---|---|---|---|---|---|---|---|---|---|---|
| unit | No | δ_{H} (J in Hz) | δ_{H} (J in Hz) | δ_{H} (J in Hz) | δ_{H} (J in Hz) | δ_{H} (J in Hz) | δ_{H} (J in Hz) | | δ_{H} (J in Hz) | δ_{H} (J in Hz) |
| Z-Dhb | 3 | 6.53 q (7.1) | 6.58 q (7.0) | — | 6.71 q (6.7) | 6.69 q (7.2) | 6.69 q (7.5) | | 6.89 d (7.8) | 6.89 d (6.6) |
| | 4 | 1.59 d (7.1) | 1.64 d (7.0) | — | 1.64 d (7.2) | 1.63 d (7.2) | 1.63 d (7.5) | | 1.73 d (7.8) | 1.73 d (6.6) |
| | NH | 8.99 s | 9.04 brs | — | 8.29 s | 8.28 s | 8.33 m | | | |
| L-Ala | 2 | 4.38 m | 4.43 m | 4.17 m | 4.28 m | 4.27 m | 4.23 m | | 4.46 m | 4.44 m |
| | 3 | 1.25 d (7.1) | 1.31 d (7.0) | 1.25 d (7.3) | 1.30 d (7.3) | 1.31 m | 1.34 d (7.0) | | 1.37 d (6.5) | 1.42 d (6.9) |
| | NH | 8.14 d (7.0) | 8.19 d (7.7) | 7.97 d (7.7) | 7.94 d (9.0) | 8.02 m | 8.33 m | | | |
| L-Val | 2 | 4.25 dd (6.7, | 4.30 dd (6.5, | 4.25 dd (7.0, | 3.89 m | 3.90 dd (7.0, | 3.96 dd (1.0, | | 4.03 m | 4.03 m |
| | | 8.2) | 8.5) | 8.7) | | 8.0) | 9.7) | | | |
| | 3 | 1.98 m | 2.03 m | 1.97 m | 1.94 m | 1.93 m | 2.01 m | | 2.04 | 2.12 m |
| | 4/5 | 0.81 d (6.7); | 0.86 d (6.0); | 0.87 d (6.6); | 0.84 d (7.0); | 0.83 d (6.9); | 0.87 d (6.7); | | 0.96 d (6.5); | 0.97 d (7.0); |
| | | 0.84 d (6.7) | 0.88 d (6.0) | 0.83 d (6.6) | 0.75 d (7.0) | 0.76 d (6.9) | 0.83 d (6.7) | | 0.95 d (6.5) | 0.95 d (7.0) |
| | NH | 7.70 d (8.6) | 7.75 (8.5) | 7.70 d (9.3) | 7.51 d (9.3) | 7.62 brs | 8.10 d (10.0) | | | |
| D-a-Thr | 2 | 4.30 dd (7.6, | 4.36 dd (7.0, | 4.31 dd (7.8) | 4.15 m | 4.03 d (5.6) | 4.05 m | | 4.19 brs | 4.22 brs |
| | | 7.6) | 7.2) | | | | | | | |
| | 3 | 3.78 m | 3.83 m | 3.78 m | 4.72 q (6.6) | 4.60 m | 4.51 m | | 4.57 m | 4.50 m |
| | 4 | 1.01 d (6.3) | 1.07 d (5.8) | 1.01 d (6.1) | 1.36 d (6.6) | 1.30 m | 1.30 d (6.3) | | 1.43 d (6.8) | 1.38 d (7.0) |
| | NH | 8.09 d (8.7) | 8.14 d (8.6) | 8.10 d (8.5) | 8.47 d (7.0) | 8.32 m | 8.33 m | | | |
| L-Phe | 2 | 4.58 m | 4.59 m | 4.58 m | 4.47 m | 4.41 m | 4.06 m | | 3.30 m | 3.39 t (7.9) |
| (L-Tyr) | 3 | 3.07 dd (3.5, | 3.06 dd (2.0, | 3.06 dd (3.5, | 3.05 dd (4.8, | 2.94 m, 2.70 m | 3.05 m | | 2.89 m | 2.89 m |
| | | 13.4); | 13.4); 2.71 m | 13.4); | 13.3); | | | | | |
| | | 2.70 dd | | 2.70 dd | 2.84 dd (10.0, 13.3) | | | | | |
| | | (11.5, 13.4) | | (11.5, 13.4) | | | | | | |
| | ph | 7.12~7.29 m | 7.02~7.30 m | 7.12~7.29 m | 7.07~7.29 m | 7.00 d (8.4); | 7.19~7.37 m | | 7.19~7.37 m | 7.19~7.37 m |
| | | | | | | 6.62 d (8.0) | | | | |
| | NH | 8.26 d (8.6) | 8.28 d (8.4) | 8.26 d (9.4) | 8.42 d (7.9) | 8.30 m | 8.81 d (7.0) | | | |
| D-Leu | 2 | 4.18 m | 4.20 m | 4.16 m | — | 4.20 m | — | | Propanone | Propanone |
| | 3 | 1.15 m | 1.15 m | 1.14 m | — | 1.26 m | — | 1 | 3.42 m | 3.27 m |
| | 4 | 1.16 m | 1.16 m | 1.15 m | — | 1.27 m | — | 3 | 2.14 s | 2.09 s |
| | 5/6 | 0.72 d (5.5); | 0.73 d (5.6); | 0.72 d (5.5), | — | 0.79 d (6.2); | — | 4 | 1.17 d (7.0) | 1.21 d (6.9) |
| | | 0.69 d (5.5) | 0.70 d (5.6) | 0.69 d (5.5) | | 0.75 m | | | | |
| | NH | 7.98 d (7.4) | 7.92 d (8.0) | 8.28 d (7.7) | — | 7.99 m | — | | — | — |
| D-Phe | 2 | 4.47 m | 4.48 m | 4.47 m | 4.48 m | 4.52 m | — | | — | — |
| | 3 | 2.91 d (3.4, | 2.93 dd (3.0, | 2.90 d (3.4, | 2.74 dd (4.0, | 2.97 m; 2.67 | — | | — | — |
| | | 13.9); | 13.7); 2.69 m | 13.9); | 13.4); | | | | | |
| | | 2.65 dd | | 2.65 dd | 2.52 dd (10.2, 13.4) | | | | | |
| | | (10.3, 13.9) | | (10.3, 13.9) | | | | | | |
| | ph | 7.12~7.29 m | 7.0~7.3 m | 7.12~7.29 m | 7.07-7.29 m | 7.14~7.26 m | — | | — | — |
| | NH | 8.01 d (8.2) | 7.91d (8.0) | 8.02 d (8.5) | 7.92 d (8.1) | 7.88 brs | — | | — | — |
| Fatty | 2 | 1.70 s | 1.98 m | 1.71 s | 1.70 s | 1.78 s | — | | — | — |
| acid | 3 | — | 0.82 m | | — | — | — | | | — |

^{a}data were measured in CD₃OD-d4

TABLE 2
¹³C (125 MHz) NMR Data for Nobilamides A-H (4-11) in DMSO-d6.

| | | | | | | |
|---|---|---|---|---|---|---|
| | | 4 | 5 | 6 | 7 | 8 |
| unit | No | δ_{C} (mult.) | δ_{C} (mult.) | δ_{C} (mult.) | δ_{C} (mult.) | δ_{C} (mult.) |

| | | | | | | | | | | |
|---|---|---|---|---|---|---|---|---|---|---|
| Z-Dhb | 1 | 166.1 | qC | 166.0 | qC | — | 163.2 | qC | 163.5 | qC |
| | 2 | 129.4 | qC | 128.5 | qC | — | 126.0 | qC | 126.3 | qC |
| | 3 | 133.8 | CH | 132.6 | CH | — | 133.8 | CH | 134.2 | CH |
| | 4 | 15.8 | CH₃ | 14.2 | CH₃ | — | 15.3 | CH₃ | 15.5 | CH₃ |

| L-Ala | 1 | 171.5 | qC | 171.4 | qC | 174.6 | qC | 170.1 | qC | 170.1 | qC |
|---|---|---|---|---|---|---|---|---|---|---|---|
| | 2 | 50.0 | CH | 48.7 | CH | 52.1 | CH | 50.0 | CH | 50.2 | CH |
| | 3 | 19.9 | CH₃ | 18.3 | CH₃ | 17.8 | CH₃ | 17.9 | CH₃ | 17.9 | CH₃ |
| L-Val | 1 | 171.2 | qC | 171.0 | qC | 171.8 | qC | 171.3 | qC | 171.3 | qC |
| | 2 | 59.0 | CH | 57.7 | CH | 57.7 | CH | 61.4 | CH | 61.5 | CH |
| | 3 | 32.6 | CH | 31.0 | CH | 31.5 | CH | 28.6 | CH | 29.2 | CH |
| | 4/5 | 21.3/19.8 | CH₃ | 19.5/18.3 | CH₃ | 20.1/18.9 | CH₃ | 19.5/19.4 | CH₃ | 19.8/19.5 | CH₃ |
| D-a-Thr | 1 | 170.4 | qC | 170.2 | qC | 170.3 | qC | 168.1 | qC | 168.0 | qC |
| | 2 | 60.2 | CH | 59.1 | CH | 59.1 | CH | 57.6 | CH | 58.3 | CH |
| | 3 | 68.8 | CH | 67.7 | CH | 67.9 | CH | 73.9 | CH | 73.4 | CH |
| | 4 | 21.8 | CH₃ | 20.4 | CH₃ | 20.7 | CH₃ | 17.9 | CH₃ | 17.8 | CH₃ |
| L-Phe (L-Tyr) | 1 | 171.8 | qC | 171.8 | qC | 171.3 | qC | 171.9 | qC | 172.2 | qC |
| | 2 | 55.9 | CH | 54.0 | CH | 54.7 | CH | 55.0 | CH | 55.2 | CH |
| | 3 | 39.5 | CH₂ | 38.2 | CH₂ | 38.5 | CH₂ | 38.2 | CH₂ | 37.0 | CH₂ |
| | 4 | 138.7 | qC | 138.6 | qC | 138.7 | qC | 137.7 | qC | 130.6 | qC |
| | 5, 9 | 128.7 | CH | 128.4 | CH | 128.7 | CH | 128.0 | CH | 127.9 | CH |
| | 6, 8 | 129.9 | CH | 129.7 | CH | 129.9 | CH | 129.1 | CH | 115.3 | CH |
| | 7 | 126.8 | CH | 126.7 | CH | 126.8 | CH | 126.5 | CH | 156.5 | qC |
| D-Leu | 1 | 172.4 | qC | 172.2 | qC | 172.4 | qC | — | | 172.6 | qC |
| | 2 | 53.1 | CH | 51.9 | CH | 48.3 | CH | — | | 52.1 | CH |
| | 3 | 42.7 | CH₂ | 41.3 | CH₂ | 41.8 | CH₂ | — | | 41.5 | CH₂ |
| | 4 | 25.8 | CH | 26.3 | CH | 24.8 | CH | — | | 24.2 | CH |
| | 5/6 | 23.8/24.7 | CH₃ | 22.8/22.6 | CH₃ | 23.5/22.6 | CH₃ | — | | 19.5/23.5 | CH₃ |
| D-Phe | 1 | 172.0 | qC | 171.6 | qC | 172.2 | qC | 172.0 | qC | 171.6 | qC |
| | 2 | 55.5 | CH | 54.7 | CH | 54.5 | CH | 54.6 | CH | 54.1 | CH |
| | 3 | 39.0 | CH₂ | 37.7 | CH₂ | 38.1 | CH₂ | 37.6 | CH₂ | 37.6 | CH₂ |
| | 4 | 138.6 | CH | 138.5 | qC | 138.6 | CH | 137.4 | qC | 138.2 | qC |
| | 5, 9 | 128.6 | CH | 128.0 | CH | 128.6 | CH | 128.0 | CH | 128.4 | CH |
| | 6, 8 | 129.8 | CH | 129.0 | CH | 129.8 | CH | 129.0 | CH | 130.1 | CH |
| | 7 | 126.8 | CH | 126.7 | CH | 126.8 | CH | 126.2 | CH | 126.7 | CH |
| Fatty acid | 1 | 169.9 | qC | 173.4 | qC | 169.9 | qC | 169.9 | qC | 169.7 | qC |
| | 2 | 24.3 | CH₃ | 28.6 | CH₃ | 23.3 | CH₃ | 22.9 | CH₃ | 23.2 | CH₃ |

| | | | | | | |
|---|---|---|---|---|---|---|
| 3 | — | 10.3 | CH₃ | — | — | — |

| | | 9 | | 10^{a} | | 11^{a} | |
|---|---|---|---|---|---|---|---|
| unit | No | δ_{C} (mult.) | | δ_{C} (mult.) | | δ_{C} (mult.) | |
| Z-Dhb | 1 | 163.1 | qC | 165.3 | qC | 165.3 | qC |
| | 2 | 126.2 | qC | 128.0 | qC | 127.9 | qC |
| | 3 | 134.1 | CH | 138.9 | CH | 138.7 | CH |
| | 4 | 15.2 | CH₃ | 15.7 | CH₃ | 15.7 | CH₃ |
| L-Ala | 1 | 168.7 | qC | 173.1 | qC | 173.2 | qC |
| | 2 | 50.3 | CH | 51.8 | CH | 52.0 | CH |
| | 3 | 17.8 | CH₃ | 18.6 | CH₃ | 19.1 | CH₃ |
| L-Val | 1 | 170.2 | qC | 174.8 | qC | 174.5 | qC |
| | 2 | 61.8 | CH | 64.1 | CH | 64.4 | CH |
| | 3 | 28.9 | CH | 31.3 | CH | 31.1 | CH |
| | 4/5 | 19.6/19.9 | CH₃ | 20.8/20.4 | CH₃ | 203/20.3 | CH₃ |
| D-a-Thr | 1 | 167.4 | qC | 171.4 | qC | 171.3 | qC |
| | 2 | 58.1 | CH | 59.0 | CH | 59.3 | qC |
| | 3 | 73.9 | CH | 76.4 | CH | 76.3 | CH |
| | 4 | 17.5 | CH₃ | 17.7 | CH₃ | 19.2 | CH₃ |
| L-Phe (L-Tyr) | 1 | 171.4 | qC | 177.1 | qC | 177.1 | qC |
| | 2 | 54.0 | CH | 64.9 | CH | 64.4 | CH |
| | 3 | 37.3 | CH₂ | 42.0 | CH₂ | 41.8 | CH₂ |
| | 4 | 135.1 | qC | 139.8 | qC | 139.4 | qC |
| | 5, 9 | 129.0 | CH | 130.4 | CH | 130.4 | CH |
| | 6, 8 | 129.8 | CH | 131.2 | CH | 131.2 | CH |
| | 7 | 127.8 | CH | 128.8 | CH | 128.9 | CH |

| D-Leu | 1 | — | | Propanone | | Propanone | |
|---|---|---|---|---|---|---|---|
| | 2 | — | 1 | 64.7 | CH | 64.6 | CH |
| | 3 | — | 2 | 215.3 | qC | 214.2 | qC |
| | 4 | — | 3 | 27.8 | CH₃ | 26.2 | CH₃ |
| | 5/6 | — | 4 | 19.3 | CH₃ | 17.6 | CH₃ |

| | | | | |
|---|---|---|---|---|
| D-Phe | 1 | — | — | — |
| | 2 | — | — | — |
| | 3 | — | — | — |
| | 4 | — | — | — |
| | 5, 9 | — | — | — |
| | 6, 8 | — | — | — |
| | 7 | — | — | — |
| Fatty acid | 1 | — | — | — |
| | 2 | — | — | — |
| | 3 | — | — | — |

^{a}data were measured in CD₃OD-d4

### EXAMPLES

### Example 1

### Data Collection

UV spectra were obtained using a Perkin-Elmer Lambda2 UV/vis spectrometer. IR spectra were recorded on a JASCO FT/IR-420 spectrometer. NMR data were collected using either a Varian NOVA 500 (¹H 500 MHz, ¹³C 125 MHz) NMR spectrometer with a 3 mm Nalorac MDBG probe or a Varian INOVA 600 (¹H 600 MHz, ¹³C 150 MHz) NMR spectrometer equipped with a 5 mm ¹H[¹³C, ¹⁵N] triple resonance cold probe with a z-axis gradient and utilized residual solvent signals for referencing. High-resolution mass spectra (HRMS) were obtained using a Bruker (Billerica, Mass.) APEXII FTICR mass spectrometer equipped with an actively shielded 9.4 T superconducting magnet (Magnex Scientific Ltd., UK), an external Bruker APOLLO ESI source, and a Synrad 50W CO₂ CW laser. All compounds were assessed to be >99% pure by HPLC with DAD and MS detectors.

### Example 2

### Fermentation and Extraction

Strains CN48 and CT3a were cultivated from dissected tissues of the mollusks Chicoreus nobilis and Conus tribblei, respectively, and each were individually grown at 30° C. with shaking at 200 rpm in a 10 L fermentor containing 10 L of ISP2 medium (0.2% yeast extract, 1% malt extract, 0.2% glucose, 2% NaCl). After 8 days, the broth was centrifuged and the supernatant was extracted with HP-20 resin for 4 hours. The resin was filtered through cheesecloth, washed with water to remove salts, and eluted with MeOH to yield the crude extract.

### Example 3

### Purification

The crude extract (650 mg) from Example 2 of CN48 was separated into 5 fractions (Fr1-Fr5) on a C₁₈ column using gradient elution of MeOH in H₂O (50%, 60%, 70%, 80%, 100%). Fr4 eluting in 80% MeOH was further purified by C₁₈ HPLC using 85% MeOH in H₂O to obtain compound 4 (100.0 mg) and compound 6 (4.0 mg), and two further fractions Fr4-3 and Fr4-4. Fr4-3 was further purified by C₁₈ HPLC using 37% CH₃CN in H₂O with 0.1% TFA to obtain compound 5 (2.0 mg), compound 7 (1.0 mg), and compound 8 (1.2 mg). Fr4-4 was further purified by C₁₈ HPLC using 45% CH₃CN in H₂O with 0.1% TFA to obtain compound 1 (150.0 mg). Fraction Fr5 was further purified by C₁₈ HPLC using 55% CH₃CN in H₂O with 0.1% TFA to obtain compound 2 (1.0 mg).

The crude extract (350 mg) from Example 2 of CT3a was separated into 5 fractions (Fr1-Fr5) on a C₁₈ column using gradient elution of MeOH in H₂O (20%, 40%, 60%, 70%, 80%). Fr4 eluting in 70% MeOH was further purified by C₁₈ HPLC using 39% CH₃CN in H₂O to obtain compound 9 (1.5 mg), compound 10 (2.0 mg) and compound 11 (3.0 mg). Compound 3 was obtained from fraction Fr2 by C₁₈ HPLC using 25% CH₃CN in H₂O. In addition, compounds 4 (1.0 mg) and 1 (0.4 mg) were also isolated from fraction Fr5 of CT3a.

### Example 4

### Compound 1a

Compound 1 (1.0 mg) from Example 3 in methanol (2.0 mL) was treated with a balloon of H₂ gas and 10% palladium on carbon (3 mg) overnight. The reaction mixture was filtered through silica gel, evaporated to dryness, and purified by HPLC (80% methanol in H₂O with 0.1% TFA) to give compound 1a (0.4 mg; 40% isolated yield): white solid; ¹H NMR (DMSO, 500 MHz), L-Phe: δ 4.63 (1H, m, α-H), 3.27 (2H, m, β-H), 6.96-7.27 (5H, m, Ph-H); D-Phe: δ 4.65 (1H, m, α-H), 2.68 (1H, dd, J=14.1, 5.0 Hz, β-H₁), 6.96-7.27 (5H, m, Ph-H); 4.67 (1H, dd, J=9.5, 5.2 Hz, α-H, Phe), 4.45 (1H, t, J=7.6 Hz, α-H, Leu), 2.92 (1H, dd, J=14.1, 10.0 Hz, β-H₂), 6.96-7.27 (5H, m, Ph-H); L-Val: δ 3.94 (1H, m, α-H), 2.06 (1H, m, β-H), 0.86 (3H, d, J=6.5 Hz, γ-Me), 0.79 (3H, d, J=6.5 Hz, γ-Me); But: δ 4.23 (1H, m, α-H), 1.57 (2H, m, β-H), 0.72 (3H, t, J=7.5 Hz, γ-Me); L-Ala: δ 3.76 (1H, m, α-H), 1.04 (3H, d, J=6.8 Hz, β-Me); D-Leu: δ 3.92 (1H, m, α-H), 1.22 (2H, m, β-H), 1.20 (1H, m, γ-H), 0.81 (6H, d, J=6.5 Hz, 6-Me); D-a-Thr: δ 4.20 (1H, m, α-H), 4.21 (1H, m, β-H), 1.30 (3H, d, J=6.5 Hz, γ-Me); δ 1.91 (3H, s, acetyl); ESIMS m/z 806 [M+H]⁺, 828 [M+Na]⁺.

### Example 5

### Compound 3, N-Acetyl-L-phenylalanyl-L-leucinamide

This compound from Example 3 is characterized as: a colorless solid; ¹H NMR (CDCl₃, 500 MHz) δ 7.18-7.28 (5H, m, Ph-H, Phe), 4.67 (1H, dd, J=9.5, 5.2 Hz, α-H, Phe), 4.45 (1H, t, J=7.6 Hz, α-H, Leu), 3.17 (1H, dd, J=14.1, 5.0 Hz, β-H₁, Phe), 2.85 (1H, dd, J=14.1, 10.0 Hz, β-H₂, Phe), 1.88 (3H, s, acetyl group), 1.71 (1H, m, γ-H, Leu), 1.64 (2H, m, β-H, Leu), 0.96 (3H, d, J=6.5 Hz, Me, Leu), 0.92 (3H, d, J=6.5 Hz, Me, Leu); ¹³C NMR (CDCl₃, 125 MHz) δ 174.5 (C, COOH, Leu), 172.6 (C, C═O, Phe), 171.9 (C, C═O, acetyl group), 137.3 (C, Ph-C, Phe), 129.1 (CH, Ph-CH, Phe), 128.2 (CH, Ph-CH, Phe), 126.5 (CH, Ph-CH, Phe), 54.7 (CH, α-CH, Phe), 50.9 (CH, α-CH, Leu), 40.5 (CH₂, β-CH₂, Leu), 37.7 (CH₂, β-CH₂, Phe), 24.8 (CH, γ-CH, Leu), 22.2 (CH₃, acetyl group), 21.1 (CH₃, Leu), 20.7 (CH₃, Leu). ESIMS m/z 321 [M+H]⁺.

### Example 6

### Compound 4, Nobilamide A

This compound from Example 3 is characterized as: a white solid; [α]²⁰_{D}−20 (c 0.1, DMSO); UV (MeOH)) λₘₐₓ (log ε) 210 (3.9), 243 (1.2) nm; IR (film) νₘₐₓ: 3272, 2978, 1808, 1712, 1696, 1648, 1568, 1553, 1537, 1112, 984 cm⁻¹; ¹H and ¹³C NMR, see Tables 1 and 2; HRESIMS m/z 844.4232 [M+Na]⁺ (calcd for C₄₂H₅₈N₇O₁₀Na, 844.4216, δ=−1.9 ppm).

### Example 7

### Compound 5, Nobilamide B

This compound from Example 3 is characterized as: a white solid; [α]²⁰_{D}+3 (c 0.01, DMSO); UV (MeOH) λₘₐₓ (log ε) 210 (4.0), 243 nm (1.3); IR (film) νₘₐₓ: 3276, 2923, 1753, 1692, 1630, 1568, 1553, 1538, 1107, 985 cm⁴; ¹H and ¹³C NMR, see Tables 1 and 2; HRESIMS m/z 858.4418 [M+Na]⁺ (calcd for C₄₃H₆₀N₇O₁₀Na, 858.4372, δ=−5.4 ppm).

### Example 8

### Compound 6, Nobilamide C

This compound from Example 3 is characterized as: a white solid; [α]²⁰_{D}−15 (c 0.1, DMSO); UV (MeOH) λₘₐₓ (log ε) 210 (3.9), 243 (1.1) nm; IR (film) νₘₐₓ: 3142, 2936, 1652, 1648, 1520, 1510, 1272, 1016 cm⁻¹; ¹H and ¹³C NMR, see Tables 1 and 2; HRESIMS m/z 761.3889 [M+Na]⁺ (calcd for C₃₈H₅₃N₆O₉Na, 761.3844, δ=5.9 ppm).

### Example 9

### Compound 7, Nobilamide D

This compound from Example 3 is characterized as: a white solid; [α]²⁰_{D}−24 (c 0.1, DMSO); UV (MeOH) λₘₐₓ (log ε) 210 (3.8), 255 (1.5) nm; IR (film) νₘₐₓ: 3281, 2930, 1750, 1703, 1641, 1563, 1537, 1521 1265, 984 cm⁻¹; ¹H and ¹³C NMR see Tables 1 and 2; HRESIMS m/z 713.3334 [M+Na]⁺ (calcd for C₃₆H₄₅N₆O₈Na, 713.3270, δ=−9.0 ppm).

### Example 10

### Compound 8, Nobilamide E

This compound from Example 3 is characterized as: a white solid; [α]²⁰_{D}−40 (c 0.01, DMSO); UV (MeOH) λₘₐₓ (log ε) 210 (3.9), 256 (1.1), 276 (0.8) nm; IR (film) νₘₐₓ: 3296, 3062, 2968, 1664, 1648, 1563, 1547, 1531, 1249, 1016 cm⁻¹; ¹H and ¹³C NMR, see Tables 1 and 2; HRESIMS m/z 842.4095 [M+Na]⁺ (calcd for C₄₂H₅₆N₇O₁₀Na, 842.4059, δ=4.3 ppm).

### Example 11

### Compound 9, Nobilamide F

This compound from Example 3 is characterized as: a white solid; [α]²⁰_{D}−5 (c 0.1, DMSO); UV (MeOH) λₘₐₓ (log ε) 210 (3.8), 255 (1.4) nm; IR (film) νₘₐₓ: 3124, 1703, 1672, 1688, 1452, 989 cm⁻¹; ¹H and ¹³C NMR, see Tables 1 and 2; HRESIMS m/z 502.2666 [M+H]⁺ (calcd for C₂₅H₃₆N₅O₆, 502.2660, δ=1.2 ppm).

### Example 12

### Compound 10, Nobilamide G

This compound from Example 3 is characterized as: a colorless solid; [α]²⁰_{D}−24 (c 0.1, DMSO); UV (MeOH) λₘₐₓ (log ε) 210 (4.0), 255 (1.3) nm; IR (film) νₘₐₓ: 3278, 2926, 1754 1704, 1655, 1508, 1458, 983 cm⁻¹; ¹H and ¹³C NMR, see Tables 1 and 2; HRESIMS m/z 572.3081 [M+H]⁺ (calcd for C₂₉H₄₂N₅O₇, 572.3079, δ=0.4 ppm).

### Example 13

### Compound 11, Nobilamide H

This compound from Example 3 is characterized as: a colorless solid; [α]²⁰_{D}−27 (c 0.1, DMSO); UV (MeOH) λₘₐₓ (log ε) 210 (4.0), 255 (1.4) nm; IR (film) νₘₐₓ: 3290, 2920, 1696, 1664, 1552, 1520, 1256, 1032 cm⁻¹; ¹H and ¹³C NMR, see Tables 1 and 2; HRESIMS m/z 572.3079 [M+H]⁺ (calcd for C₂₉H₄₁N₅O₇, 572.3079, δ=0 ppm).

### Example 14

### DRG Assay

DRG cells from cervical and lumbar regions were obtained from C57B1 mice. DRG cells were suspended in medium with additives and loaded with Fura-2 AM (Molecular Probes), a fluorescent dye used to measure intracellular calcium levels. More specifically, thirty nine three-week old C57B1/6 mice were anesthetized with isoflurane, and euthanized by cervical dislocation. Dorsal root ganglia (DRG) from cervical and lumbar regions were removed into HBSS, treated with trypsin, washed with MEM (Invitrogen) containing 10% FBS (Hyclone), 2.4% glucose (Sigma), 1% glutamax (Invitrogen) and 1% penicillin/streptomycin (Atlanta Biological). DRGs were triturated, pre-plated, and incubated at 37° C. for 1 h to reduce non-neuronal cells. Neurons were gently swirled and centrifuged (110×g, 5 min). Cells were removed, re-suspended and 30 μL plated onto the 4 center wells of 24-well plates coated with poly-L-lysine and laminin. A 0.5 mm-thick silicone ring with a 4 mm-diameter opening sealed to the surface of each well reduced the plating area in each well. After one hour, wells were filled with 37° C. culture medium with 10 ng/mL GDNF (Glial Derived Neurotrophic Factor; Preprotech). Sixteen to twenty three hours after plating, cultures were loaded with Fura-2 AM (Molecular Probes) fluorescent dye for 40-60 min, then washed with pH 7.4 oxygenated observation medium containing no additional ATP (145 mM NaCl, 5 mM KCl, 2 mM CaCl2, 1 mM MgCl2, 1 mM citrate, 10 mM glucose, 10 mM MES, 10 mM HEPES). The final well volume was 500 μL. KCl solution was made from observation medium with an additional 25 mM KCl (KCl-Obs). Extracts from cultivated isolates (˜0.5 mg each) were dissolved in a minimum amount of DMSO (approx 10 μL), then 5 μL of this solution was diluted in observation medium (1 mL), and 5 μL was diluted in KCl-Obs (1 mL). As the most concentrated observed metabolites in the extracts comprised ˜1% of the extract weight, this allowed estimation of a maximum concentration of 10 μM for the most abundant extracts in the assay. For each experiment approximately 30 images were acquired as a baseline control period prior to the addition of experimental solutions. Experiments were performed at room temperature (20 to 25° C.) in a 24-well plate format using fluorescence microscopy. Individual cells were treated as single samples, so that the individual responses of diverse neuron subtypes from the DRG could be examined. After baseline measurements, the cells were treated with 25 mM KCl solution and then washed. After return to baseline, bacterial extracts, fractions, or pure compounds were applied. This solution was then later replaced with 25 mM KCl solution 5 min later. To differentiate pain-sensing and TRPV1-expressing neurons from other neuronal types, capsaicin was applied after return to baseline of the extracts. Finally, additional pulses of 25 mM KCl or 100 mM KCl were applied to determine whether cells were still viable with normal action potentials.

In long-term TRPV1 antagonism test, the cells were treated with capsaicin solution (100 or 200 nM) and then washed. After fluorescence returned to baseline, test compounds (125 μM) were applied and incubated for 5 min. The solution was decanted, and capsaicin solution (100 or 200 nM) was added, followed by a wash. After 3 hours, another capsaicin solution was applied. Finally, an additional pulse of KCl (100 mM) was applied to determine whether cells were still viable with normal action potentials. To test for protein synthesis and trafficking, cycloheximide (10 ug/ml), actinomycin D (6.3 ug/ml) and brefeldin A (5.6 ug/ml) were added either before or after compound application.

### Example 15

### TRPV1 Assays

Cell-line based assays were performed using a BMG Labtech NOVOStar fluorescence plate reader equipped with a plate-to-plate reagent delivery system. Human immortalized bronchial epithelial (BEAS-2B) cells that stably overexpress human TRPV1 primarily intracellularly on the endoplasmic reticulum and exhibit an EC₅₀ for capsaicin-induced calcium flux of 1-2 μM. Transiently transfected human embryonic kidney (HEK-293) cells were used for experiments involving overexpressed TRPV1 and mutants thereof. In these cells, TRPV1 is expressed primarily on the cell membrane.

BEAS-2B cells were grown to confluence in fibronectin/collagen/albumin coated 96-well plates in LHC-9 growth media. Cells were prepared for the calcium flux assay by replacing the growth media with a 1:1 solution of LHC-9 and Fluo 4-Direct (Invitrogen) reagent containing Fluo 4-AM, pluronic F-127, probenecid, and a proprietary quencher dye. Cells were incubated at room temperature (˜22° C.) for 1 h in the dark and subsequently washed by replacing the loading solution with LHC-9 containing 1 mM water-soluble probenecid (Invitrogen) and 750 μM Trypan Red (ATT Bioquest). For pre-incubation experiments, test compounds were then added to the wash solutions in varying concentrations. After 30 min incubation of cells at room temperature, assays were initiated by addition of capsaicin to a final concentration of 2.5 μM at 37° C. (or 25 μM for HEK-293 cells). If test compounds were not pre-incubated, they were added concurrently with capsaicin. Changes in intracellular fluorescence (resulting from changes in cytosolic Ca²⁺) were monitored for 1 min. Data were quantified in two ways. Rates were (ΔF/sec) determined in comparison to the initial linear response observed in a control consisting of capsaicin only-treated cells. The magnitude of the response (ΔFmax) observed for the entire 1 min time period was also calculated in comparison to control.

Human TRPV1 was cloned into the pcDNA3.1D V5/His vector (Invitrogen) and modified using the QuickChange site-directed mutagenesis kit (Stratagene). Mutations were confirmed by DNA sequencing. Plasmids (200 ng/well) were transfected into HEK-293 cells grown to confluence in 1% gelatin-coated 96-well plates using Lipofectamine 2000 (2:1 lipid:DNA ratio) prepared in OptiMEM media. Cells were cultured in the presence of the reagent for 4 h and cultured for 48 h in DMEM:F12+5% FBS. After this time cells were processed and assayed for calcium flux as described above, except that the fluorophore loading steps were performed at 37° C.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A TRPV1 channel antagonist having the structure: 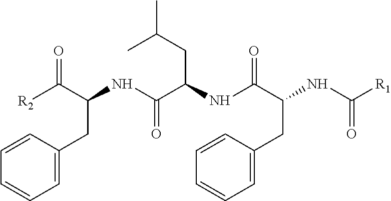
wherein R₁=—CH₃, —(CH₂)ₓ(CH)_{y}CH₃ where x+y=1-20, an aromatic, a (CH₂)ₙ aromatic where n is less than or equal to 6, a lipid, or a linker;
wherein R₂ is 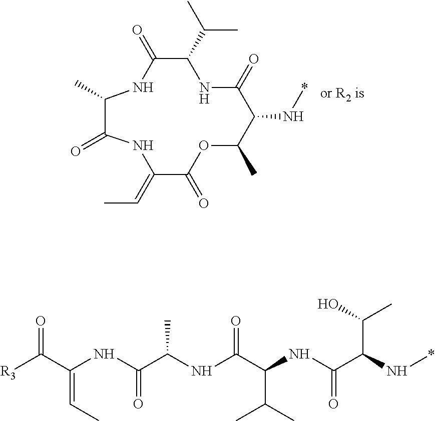
wherein R₃ is —O—R₄ or —NH—R₄; and
wherein R₄ is —H, —CH₃, an ester, a cyclic ester, or an amide.

2. The TRPV1 channel antagonist of claim 1 dispersed in a physiologically acceptable carrier.

3. The TRPV1 channel antagonist of claim 2, wherein the TRPV1 channel antagonist is present in the physiologically acceptable carrier at a concentration of from about 10 to about 1000 micromolar.

4. The TRPV1 channel antagonist of claim 1, wherein R₁ is selected from the group consisting of methyl, ethyl, propyl, isopropyl, butyl, isobutyl, tert-butyl, pentanoyl, hexanoyl, heptanoyl, octanoyl, nonanoyl, decanoyl, lauryl, myristyl, palmitoyl, stearoyl, palmitoleoyl, stearoyl, arachidonoyl, isoprenyl, farnesyl, geranyl, angelyl, aminomethyl, hydroxymethyl, thiomethyl, aminoethyl, hydroxyethyl, thioethyl, aminobutyl, hydroxybutyl, thiobutyl. Non-limiting examples of alkyl aromatic groups can include benzyl, phenyl, biphenyl, triphenyl, indolyl, furanyl, thiophenyl, pyridinyl, pyranyl, bypyridyl, imidazolyl, triazolyl, napthylenyl, and combinations thereof.

5. The TRPV1 channel antagonist of claim 1, wherein R₁ is a lipid selected from the group consisting of methyl, ethyl, propyl, benzyl, cypionyl, phenyl, aromatic, hydroxyethyl, and combinations thereof.

6. The TRPV1 channel antagonist of claim 1, wherein R₁ is a linker selected from the group consisting of polyethylene glycol, polyamines, ethanolamines, alkyl, spermidine, and combinations thereof.

7. The TRPV1 channel antagonist of claim 1, having the structure:

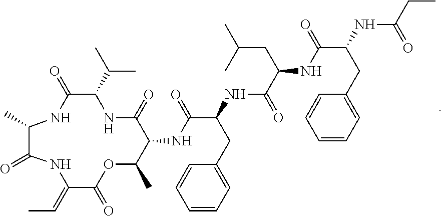 [SMILES: C/C=C1\NC(=O)[C@H](C)NC(=O)[C@H](C(C)C)NC(=O)[C@H](NC(=O)[C@H](Cc2ccccc2)NC(=O)[C@@H](CC(C)C)NC(=O)[C@@H](Cc2ccccc2)NC(=O)CC)[C@@H](C)OC1=O]

8. The TRPV1 channel antagonist of claim 1, having the structure:

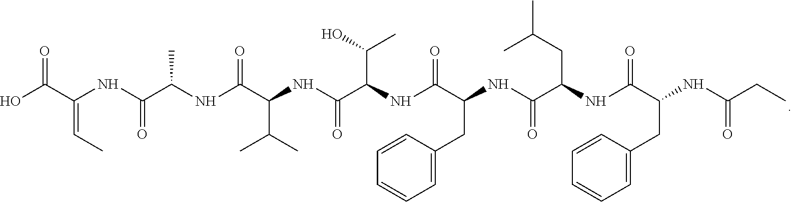 [SMILES: C/C=C(\NC(=O)[C@H](C)NC(=O)[C@@H](NC(=O)[C@H](NC(=O)[C@H](Cc1ccccc1)NC(=O)[C@@H](CC(C)C)NC(=O)[C@@H](Cc1ccccc1)NC(=O)CC)[C@@H](C)O)C(C)C)C(=O)O]

9. A method of antagonistically blocking a TRPV1 channel, comprising delivering a TRPV1 channel antagonist to the TRPV1 channel, wherein the TRPV1 channel antagonist has the structure: 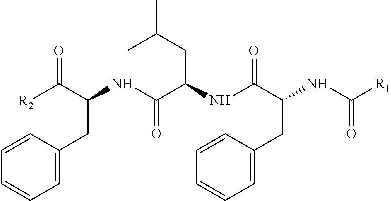
wherein R₁=—CH₃, —CH₂CH₃, —CH₂CH₂CH₃, —CH₂CH₂CH₂CH₃, —CH₂CH₂CH₂CH₂CH₃, —CH₂CH₂CH₂CH₂CH₂CH₃, a lipid, or a [linker];
wherein R₂ is 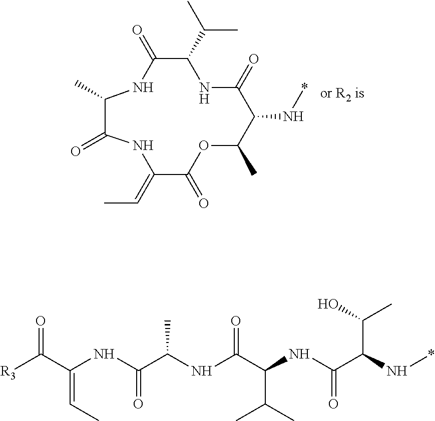
wherein R₃ is —O—R₄ or —NH—R₄; and
wherein R₄ is —H, —CH₃, an ester, a cyclic ester, or an amide.

10. The method of claim 9, wherein the TRPV1 channel is located in vivo in or on a subject, and the TRPV1 channel antagonist is delivered to the subject.

11. The method of claim 10, wherein the subject is a human.

12. The method of claim 10, wherein the TRPV1 channel antagonist is delivered to the subject in a dosage of from about 10 to about 1000 micromolar.

13. The method of claim 11, wherein the TRPV1 channel antagonist is delivered to the subject in a dosage form selected from the group consisting of topical, oral, intravenous, intramuscular, subcutaneous, buccal, ocular, nasal, and combinations thereof.

14. The method of claim 11, wherein the TRPV1 channel antagonist is delivered to the subject topically.

15. The method of claim 11, wherein the TRPV1 channel antagonist is delivered to the subject following a painful stimulus.

16. The method of claim 15, wherein the painful stimulus is capsaicin.

17. The method of claim 11, wherein the TRPV1 channel antagonist is delivered to the subject prior to a painful stimulus.

18. The method of claim 17, wherein the painful stimulus is capsaicin.

19. A method of protecting sensitive areas of a human from effects associated with a TRPV1 channel agonist exposure, comprising applying to the sensitive areas a composition comprising the TRPV1 channel antagonist of claim 1 in a physiologically acceptable carrier.

20. The method of claim 19, wherein the TRPV1 channel agonist is capsaicin.
